(19) United States
(12) Patent Application Publication
(10) Pub. No.: US 2025/0225656 A1
(43) Pub. Date: Jul. 10, 2025

(54) SYSTEMS AND METHODS FOR DETERMINING VITREOUS HAZE

(71) Applicant: REGENERON PHARMACEUTICALS, INC., Tarrytown, NY (US)

(72) Inventors: Farshid SEPEHRBAND, Scarsdale, NY (US); Karl LANDHEER, White Plains, NY (US)

(73) Assignee: REGENERON PHARMACEUTICALS, INC., Tarrytown, NY (US)

(21) Appl. No.: 18/853,045

(22) PCT Filed: Mar. 30, 2023
(86) PCT No.: PCT/US2023/065123

§ 371 Date: Sep. 30, 2024

Related U.S. Application Data
(60) Provisional application No. 63/453,718, filed on Mar. 21, 2023.
(60) Provisional application No. 63/325,722, filed on Mar. 31, 2022.

(51) Int. Cl. G06T 7/00; A61B 3/12; G06T 5/10; G06T 5/20; G06T 5/40; G06T 5/70; G06T 5/92; G06T 7/90

(52) U.S. Cl. CPC G06T 7/0012; A61B 3/12; G06T 5/10; G06T 5/20; G06T 5/40; G06T 5/70; G06T 5/92; G06T 7/90; G06T 2207/10024; G06T 2207/20056; G06T 2207/30041; G06T 2207/30168

(57) ABSTRACT

An example embodiment of the present disclosure provides systems and methods for grading vitreous haze. A color fundoscopic photograph may be obtained. A color channel of the color fundoscopic photograph may be isolated. The color channel of the color fundoscopic photograph may be normalized. A window function may be applied to the normalized color channel to obtain a windowed color channel. A smoothing function may be applied to the windowed color channel to obtain a smoothed color channel. A high-pass filter may be applied to the smoothed color channel to obtain a filtered color channel. The filtered color channel may be transformed to a frequency domain from a spatial domain. A magnitude spectrum may be calculated. The magnitude spectrum may be integrated to determine a clarity score. A haziness score may be calculated based on the clarity score.

### CROSS-REFERENCE TO RELATED APPLICATIONS

This application is a Patent Cooperation Treaty (PCT) application of, and claims priority under Article 8 of the PCT to U.S. Provisional Patent Application No. 63/453,718, filed Mar. 21, 2023, entitled, “SYSTEMS AND METHODS FOR DETERMINING VITREOUS HAZE” and U.S. Provisional Patent Application No. 63/325,722, filed Mar. 31, 2022, entitled, “SYSTEMS AND METHODS FOR DETERMINING VITREOUS HAZE,” the entire contents of each of which are fully incorporated herein by reference.

### FIELD OF THE DISCLOSURE

The various embodiments of the present disclosure relate generally to systems and methods of quantifying the blurriness of ocular fundus images. More particularly, this disclosure relates to systems and methods for automating the quantification of blurriness in standard fundoscopic images.

### BACKGROUND

Intraocular inflammatory processes lead to progressive accumulation of cells and protein exudate in the vitreous, often referred to as vitreous haze. Vitreous haze is used as a marker for intraocular inflammation caused by a variety of inflammatory diseases. Photographic scales have been introduced to standardize the grading of vitreous haze in a clinical setting, including the widely used nine-point Miami scale. Attempts to automate the grading process of vitreous haze such as the Passaglia quantitative vitreous haze algorithm (“PQVHA”), suffer from poor performance when compared to human expert graders.

Accordingly, the present disclosure is directed to systems and methods for automating the quantification of vitreous haze in fundoscopic images without sacrificing grading accuracy.

### SUMMARY

The present disclosure provides systems and methods for determining vitreous haze in a subject. The present disclosure provides for a methodology termed Robust Quantitative Vitreous Haze Algorithm (“RQVHA”). The present method may include obtaining a color funduscopic photograph. The present method may include isolating a color channel of the color fundoscopic photograph. The present method may include normalizing the color channel of the color fundoscopic photograph. The present method may include applying a window function to the normalized color channel to obtain a windowed color channel. The present method may include applying a smoothing function to the windowed color channel to obtain a smoothed color channel. The present method may include applying a high-pass filter to the smoothed color channel to obtain a filtered color channel. The present method may include transforming the filtered color channel from a spatial domain to a frequency domain. The present method may include calculating a magnitude spectrum based on the frequency domain. The method present may include integrating over the magnitude spectrum to determine a clarity score. The present method may include calculating a haziness score based on the clarity score.

In another aspect, a method for grading vitreous haze is disclosed. The method may include obtaining a color fundoscopic photograph. The method may include isolating a color channel of the color fundoscopic photograph. The method may include normalizing the color channel of the color fundoscopic photograph. The method may include applying a high-pass filter to the smoothed color channel to obtain a filtered color channel. The method may include transforming the filtered color channel from a spatial domain to a frequency domain. The method may include integrating over the magnitude spectrum to determine a clarity score. The method may include calculating a haziness score based on the clarity score.

In another aspect, a system for grading vitreous haze is disclosed. The system may include one or processors and a non-transitory memory storing instructions, that when executed by the one or more processors are configured to cause the one or more processors to perform steps of a method. The method may include, obtaining a color fundoscopic photograph. The method may include isolating a color channel of the color fundoscopic photograph. The method may include normalizing the color channel of the color fundoscopic photograph. The method may include applying a window function to the normalized color channel to obtain a windowed color channel. The method can include applying a smoothing function to the windowed color channel to obtain a smoothed color channel. The method can include applying a high-pass filter to the smoothed color channel to obtain a filtered color channel. The method can include transforming the filtered color channel from a spatial domain to a frequency domain. The method may include calculating a magnitude spectrum based on the frequency domain. The method may include integrating over the magnitude spectrum to determine a clarity score. The method may include calculating a haziness score based on the clarity score.

In another aspect, a method for processing an imaging is disclosed. A fundoscopic image can be obtained. A window function can be applied to the fundoscopic image to obtain a smoothed fundoscopic image. The smoothed fundoscopic image can be filtered with a high-pass filter to obtain a filtered fundoscopic image. The filtered fundoscopic image can be transformed from a spatial domain to a frequency domain. A magnitude spectrum can be calculated based on the frequency domain. A haziness score of the fundoscopic image can be determined based on the calculated magnitude spectrum.

These and other aspects of the present disclosure are described in the Detailed Description below and the accompanying drawings. Other aspects and features of embodiments will become apparent to those of ordinary skill in the art upon reviewing the following description of specific, example embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while example embodiments may be discussed below as device, system, or method embodiments, it is to be understood that such example embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 is a block diagram of an example system that may be used to algorithmically grade vitreous haze in collected fundoscopic images, in accordance with an example embodiment of the present disclosure.

FIG. 2 is a block diagram of an example vitreous haze grading platform as shown in FIG. 1 with additional details.

FIGS. 3A-3F provides an overview of an example RQVHA methodology, in accordance with an example embodiment of the present disclosure.

FIGS. 4A and 4B show semi-log histograms of the coefficient of variation for RQHVA and PQVHA, respectively, across images corresponding to a respective eye of the same subject, in accordance with an example embodiment of the present disclosure.

FIG. 4C and FIG. 4D show raw color fundoscopic image pairs which yielded the largest vitreous haze range for the RQVHA methodology, in accordance with an example embodiment of the present disclosure.

FIG. 4E and FIG. 4F show images corresponding to FIG. 4C and FIG. 4D, respectively, but processed with contrast limited adaptive histogram equalization for display purposes, in accordance with an example embodiment of the present disclosure.

FIG. 5A depicts a histogram comparing the haze scores obtained by the RQVHA methodology to a first expert rater across all image sets, in accordance with an example embodiment of the present disclosure.

FIG. 5B depicts a histogram comparing the haze scores obtained by the RQVHA methodology to a second expert rater across all image sets, in accordance with an example embodiment of the present disclosure.

FIG. 5C depicts a histogram comparing the haze scores obtained by the PQVHA methodology to a first expert rater across all image sets, in accordance with an example embodiment of the present disclosure.

FIG. 5D depicts a histogram comparing the haze scores obtained by the PQVHA methodology to a second expert rater across all image sets, in accordance with an example embodiment of the present disclosure.

FIG. 5E depicts a histogram comparing the haze scores obtained by the second expert rater to the first expert rater across all image sets, in accordance with an example embodiment of the present disclosure.

FIG. 5F depicts a histogram comparing the haze scores obtained by the RQVHA methodology to the PQVHA methodology across all image sets, in accordance with an example embodiment of the present disclosure.

FIG. 6A depicts haze score variation for a patient that exhibited the largest decrease in haze over time for the RQVHA methodology, the PQVHA methodology, and as observed by both expert raters, in accordance with an example embodiment of the present disclosure.

FIG. 6B depicts haze score variation for a patient that exhibited the largest difference in haze over time for the RQVHA methodology, the PQVHA methodology, and as observed by both expert raters, in accordance with an example embodiment of the present disclosure.

FIG. 6C shows the images associated with FIG. 6A but processed with contrast limited adaptive histogram equalization for display purposes, in accordance with an example embodiment of the present disclosure.

FIG. 6D shows the images associated with FIG. 6B but processed with contrast limited adaptive histogram equalization for display purposes, in accordance with an example embodiment of the present disclosure.

FIG. 7A depicts a raw color fundus photograph which includes an artifact corresponding to a dust particle on the lens of the camera, in accordance with an example embodiment of the present disclosure.

FIG. 7B depicts a cropped grayscale, contrast-inverted, and mean-filtered image corresponding to the image of FIG. 7A, in accordance with an example embodiment of the present disclosure.

FIG. 7C depicts a local entropy image corresponding to FIGS. 7A and 7B, in accordance with an example embodiment of the present disclosure.

FIG. 8 is a flowchart of an example method of calculating a haze score using the RQVHA methodology, in accordance with an example embodiment of the present disclosure.

FIGS. 9A and 9B show semi-log histograms of ranges for RQHVA and standard deviation of RQHVA, across images corresponding to a respective eye of the same subject, in accordance with an example embodiment of the present disclosure.

FIG. 9C and FIG. 9D show raw color fundoscopic image pairs which yielded the largest vitreous haze range for the RQVHA methodology, in accordance with an example embodiment of the present disclosure.

FIG. 9E and FIG. 9F show successive images corresponding to FIG. 9C and FIG. 9D, respectively, and correspond the toe second largest RQVHA range, in accordance with an example embodiment of the present disclosure.

FIG. 10A depicts a histogram comparing the haze scores obtained by the RQVHA methodology to a first expert rater across all image sets using three comparison metrics (s, κ, and τ), in accordance with an example embodiment of the present disclosure.

FIG. 10B depicts a histogram comparing the haze scores obtained by the RQVHA methodology to a second expert rater across all image sets using three comparison metrics (s, κ, and τ), in accordance with an example embodiment of the present disclosure.

FIG. 10C depicts a histogram comparing the haze scores obtained by the PQVHA methodology to a first expert rater across all image sets using three comparison metrics (s, κ, and τ), in accordance with an example embodiment of the present disclosure.

FIG. 10D depicts a histogram comparing the haze scores obtained by the PQVHA methodology to a second expert rater across all image sets using three comparison metrics (s, κ, and τ), in accordance with an example embodiment of the present disclosure.

FIG. 10E depicts a histogram comparing the haze scores obtained by the second expert rater to the first expert rater across all image sets using three comparison metrics (s, κ, and τ), in accordance with an example embodiment of the present disclosure.

FIG. 10F depicts a histogram comparing the haze scores obtained by the RQVHA methodology to the PQVHA methodology across all image sets using three comparison metrics (s, κ, and τ), in accordance with an example embodiment of the present disclosure.

FIG. 11A depicts a raw color fundus photograph which includes, in accordance with an example embodiment of the present disclosure.

FIG. 11B depicts a cropped grayscale, contrast-inverted, and mean-filtered image corresponding to the image of FIG. 11A, in accordance with an example embodiment of the present disclosure.

FIG. 11C depicts a local entropy image corresponding to FIGS. 11A and 11B, in accordance with an example embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Where values are described as ranges, it will be understood that such value includes the values of all possible sub-ranges within such ranges, as well as specific numerical values that fall within such ranges irrespective of whether a specific numerical value or specific sub-range is expressly stated. In addition, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “photograph,” as used herein, can be used interchangeably with the term “image.” Both the term “photograph” and “image” can refer to fundoscopic images used to assess the health and condition of the eye of a patient.

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods.

Vitreous haze can be used as a primary outcome for clinical trials studying a variety of ocular disorders. The severity of vitreous haze can change over the course of disease. Vitreous haze can be characterized by decreased visibility of the retinal vasculature, optic nerve head, and other fundus details revealed by ophthalmoscopic inspection of the eye. Accordingly, the present disclosure can be used as a method of diagnosis of a variety of ocular disorders that are characterized by the presence of vitreous haze. For example, the disclosed technology can be used to diagnose conditions not limited to but including uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, and reticulum cell sarcoma.

Reference will now be made in detail to example embodiments of the disclosed technology that are illustrated in the accompanying drawings and disclosed herein. Wherever convenient, the same reference numbers will be used throughout the drawings to refer to the same or like parts.

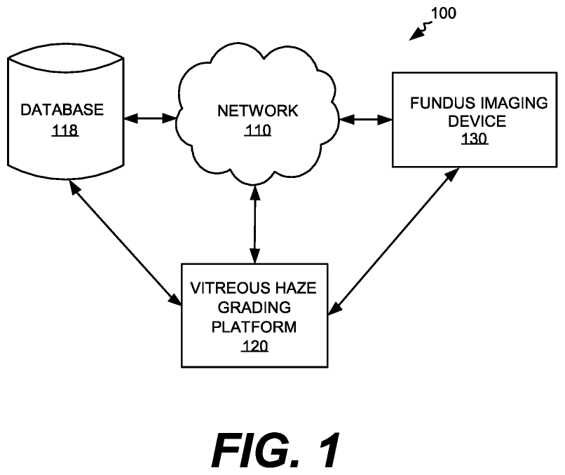

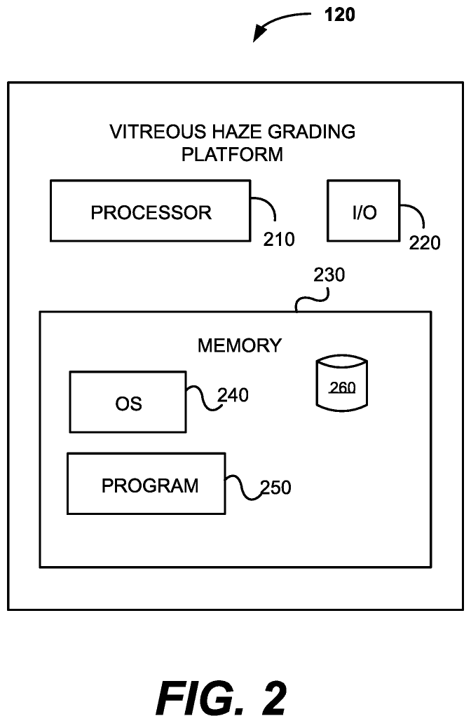

In accordance with certain disclosed embodiments, and as shown in FIG. 1, system environment 100 may include a vitreous haze grading platform 120 in communication with a fundus imaging device 130, and a database 118 over network 110. Vitreous haze grading platform 120 may be a computing device, such as a mobile computing device (e.g., a smart phone, tablet computer, smart wearable device, portable laptop computer, voice command device, wearable augmented reality device, or other mobile computing device or fixed computing device (e.g., a desktop computer or server). An example architecture of tissue imaging platform 120 that may be used to implement one or more aspects of system 100 is described below with reference to FIG. 2.

Vitreous haze grading platform 120 may receive imaging data, such as color fundoscopic photographs, that vitreous haze grading platform 120 may use to determine vitreous haze based on the received imaging data.

Fundus imaging device 130 may be an image capture device configured to capture color fundus photographs of the eye of a patient. According to some embodiments, the fundus imaging device 130 may be a specialized digital camera that utilizes indirect ophthalmoscopy to capture digital fundus images of an eye. In some embodiments, the fundus imaging device 130 may be a mobile device with a digital camera in combination with a specialized lens that enables the mobile device to capture high quality fundoscopic images of the eye. Spatial transcriptomics platform 130 may be a computing device, such as a mobile computing device (e.g., a smart phone, tablet computer, smart wearable device, portable laptop computer, voice command device, wearable augmented reality device, or other mobile computing device or fixed computing device (e.g., a desktop computer or server).

According to some embodiments, the database 118 may be a database that stores fundoscopic images to be graded by vitreous haze grading platform 120. The database 118 may also serve as a back-up storage device and may contain data and information that is also stored on, for example, database 280, as will be discussed with reference to FIG. 2. The database 118 may be accessed by the vitreous hazing grading platform 120 and may be used to determine levels of vitreous haze associated with the images.

Network 110 may be of any suitable type, including individual connections via the internet such as cellular or Wi-Fi networks. In some embodiments, network 110 may connect terminals using direct connections such as radio-frequency identification (RFID), near-field communication (NFC), Bluetooth™, low-energy Bluetooth™ (BLE), Wi-Fi™, ZigBee™, ambient backscatter communications (ABC) protocols, USB, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security. One of ordinary skill will recognize that various changes and modifications may be made to system environment 100 while remaining within the scope of the present disclosure. Moreover, while the various components have been discussed as distinct elements, this is merely an example, and, in some cases, various elements may be combined into one or more physical or logical systems. According to some embodiments, database 118, vitreous haze grading platform 120, and fundus imaging device 130 may be directly connected in lieu of, or in addition to being in communication via network 110.

FIG. 2 is a block diagram (with additional details) of the vitreous haze grading platform 120, as also depicted in FIG. 1. According to some embodiments, fundus imaging device platform 130 and database 118 may have a similar structure and components that are similar to those described with respect to vitreous haze grading platform 120 shown in FIG. 2. As shown, the vitreous haze grading platform 120 may include a processor 210, an input/output (“I/O”) device 220, a memory 230 containing an operating system (“OS”) 240 and a program 250. In certain example implementations, the vitreous haze grading platform 120 may be a single server or may be configured as a distributed computer system including multiple servers or computers that interoperate to perform one or more of the processes and functionalities associated with the disclosed embodiments. In some embodiments, the vitreous haze grading platform 120 may further include a peripheral interface, a transceiver, a mobile network interface in communication with the processor 210, a bus configured to facilitate communication between the various components of the vitreous haze grading platform 120, and a power source configured to power one or more components of the vitreous haze grading platform 120.

A peripheral interface, for example, may include the hardware, firmware and/or software that allows communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™ low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 210 to communicate with other devices via wired or wireless networks, whether local or wide area, private or public, as known in the art. A power source may be configured to provide an appropriate alternating current (AC) or direct current (DC) to power components.

The processor 210 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 210 may be one or more known processing devices, such as, but not limited to, a microprocessor from the Pentium™ family manufactured by Intel™ or the Turion™ family manufactured by AMD™. The processor 210 may constitute a single core or multiple core processor that executes parallel processes simultaneously. For example, the processor 210 may be a single core processor that is configured with virtual processing technologies. In certain embodiments, the processor 210 may use logical processors to simultaneously execute and control multiple processes. The processor 210 may implement virtual machine technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

In accordance with certain example implementations of the disclosed technology, the vitreous haze grading platform 120 may include one or more storage devices configured to store information used by the processor 210 (or other components) to perform certain functions related to the disclosed embodiments. In one example, the vitreous haze grading platform 120 may include the memory 230 that includes instructions to enable the processor 210 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the vitreous haze grading platform 120 may include a memory 230 that includes instructions that, when executed by the processor 210, perform one or more processes consistent with the functionalities disclosed herein. Methods, systems, and articles of manufacture consistent with disclosed embodiments are not limited to separate programs or computers configured to perform dedicated tasks. For example, the vitreous haze grading platform 120 may include the memory 230 that may include one or more programs 250 to perform one or more functions of the disclosed embodiments.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 210, perform one or more processes consistent with the disclosed embodiments. In some embodiments, the memory 230 may include a database 280 for fundoscopic images used by vitreous haze grading platform 120 to autonomously determine a vitreous haze score for color fundoscopic images.

The vitreous haze grading platform 120 may also be communicatively connected to one or more memory devices (e.g., databases) locally or through a network. The remote memory devices may be configured to store information and may be accessed and/or managed by the vitreous haze grading platform 120. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The vitreous haze grading platform 120 may also include one or more I/O devices 220 that may comprise one or more interfaces for receiving signals or input from devices and providing signals or output to one or more devices that allow data to be received and/or transmitted by the vitreous haze grading platform 120. For example, the vitreous haze grading platform 120 may include interface components, which may provide interfaces to one or more input devices, such as one or more keyboards, mouse devices, touch screens, track pads, trackballs, scroll wheels, digital cameras, microphones, sensors, and the like, that enable the vitreous haze grading platform 120 to perform aspects consistent with the disclosure.

In example embodiments of the disclosed technology, the vitreous haze grading platform 120 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the vitreous haze grading platform 120 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the vitreous haze grading platform 120 may include a greater or lesser number of components than those illustrated.

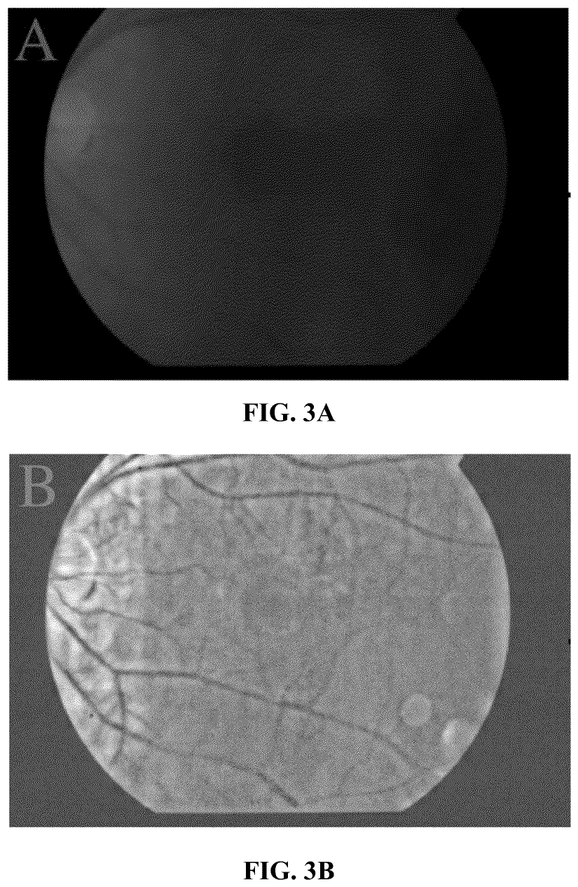

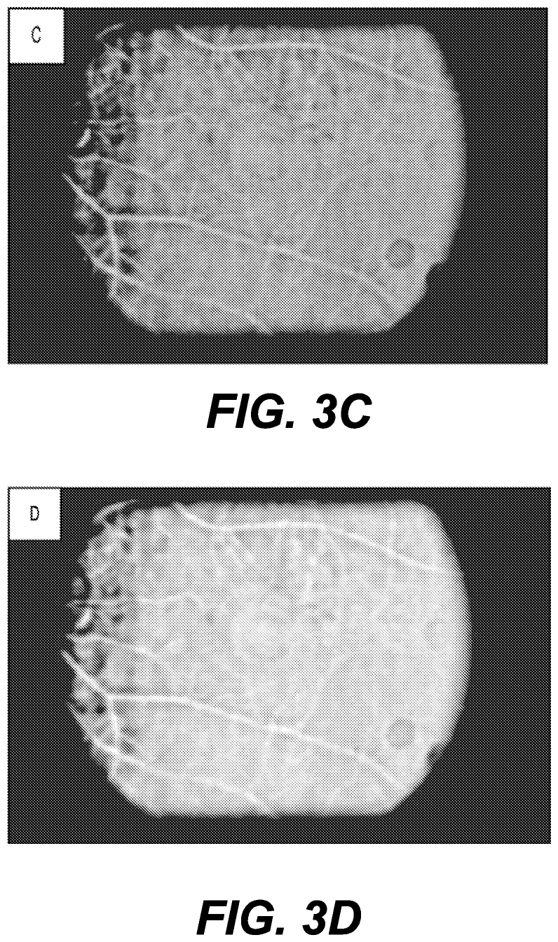

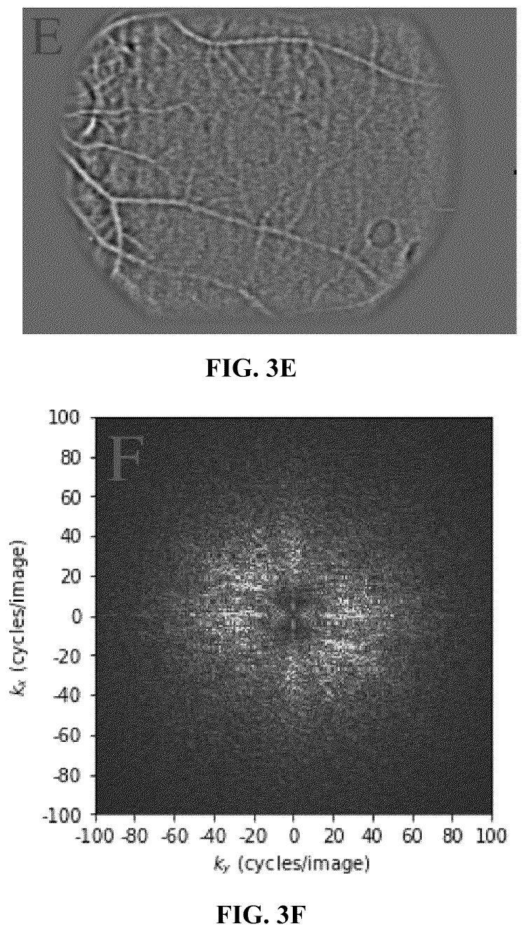

FIG. 3A shows a raw color fundus photograph as stored in database 118 and/or captured by fundus imaging device 120. The RQVHA methodology begins with receiving the raw color fundus photograph. After receiving the raw color fundus photograph as depicted in FIG. 3A from either database 118 or fundus imaging device 130, the vitreous haze grading platform 120 may isolate a single color channel from the color fundus photograph. In some embodiments, the vitreous haze grading platform 120 may isolate the green color channel because the green color channel can have a higher contrast ratio than other color channels (e.g., red or blue) present in the raw color fundus photograph. After the green channel has been isolated from the raw color fundus photograph, the vitreous haze grading platform may apply a contrast limited adaptive histogram equalization function and a gamma correction to adjust for differences in lighting in between images captured under varying light conditions. The equation representing these transformations is found below as Equation (1).

$${}^{B} I ( x , y ) = γ \{ C [ G ( {}^{A} I ( x , y ) ) ] \} , \qquad ( 1 )$$

In Equation (1), the term ^{A}I(x, y) represents the raw fundus photograph, G is the green function (i.e., a function that isolates the green channel from the fundus photograph), C is the contrast limited adaptive histogram equalization function, and γ is the gamma correction function. Superscript A corresponds to FIG. 3A and superscript B corresponds to FIG. 3B. In some embodiments, adaptive histogram equalization may be used instead of contrast limited adaptive histogram equalization. In some embodiments, any channel (e.g., red, or blue) can be isolated in place of the green channel, or the image can be converted to grayscale rather than isolating a channel. According to some embodiments the contrast limited adaptive histogram equalization function can include various parameters, which can be selected to optimize the resultant image. In some embodiments, the parameters such as the clip limit and the tile size can be set for the contrast limited adaptive histogram equalization function. Contrast limited adaptive histogram equalization is a variation of adaptive histogram equalization that limits the amplification of contrast by clipping the histogram at a predefined value before computing a cumulative distribution function, which limits the problem of noise amplification. In a preferred embodiment, the clip limit may be set to 20, and the tile size may be set to 36 by 36 pixels, but the clip limit may be varied from 2 to 100, and the tile size may be varied between 4 by 4 pixels and 128 by 128 pixels. The gamma correction function can also include a gamma value parameter which can be varied. In preferred embodiments, the gamma value can be set to 2, although the gamma value parameter can be varied between 1 and 4 in some embodiments.

FIG. 3B depicts the color fundus photograph of FIG. 3A after the green color channel has been isolated, the contrast limited adaptive histogram equalization and the gamma correction function have been applied to the image. While features of the fundus cannot be readily seen in FIG. 3A, the transformations applied to the image of FIG. 3B have increased the contrast of the features of the fundus to increase their visibility.

The next step of RQVHA methodology can involve reversing the contrast of the image and applying a window function across the image. For example, the features that are dark in FIG. 3B (e.g., the blood vasculature of the eye) will become bright in FIG. 3C when the contrast of the image is reversed. In addition, a window function can be applied to the image in order to avoid capturing sharp boundaries of the macula that are present within color fundus images. When using frequency-based algorithms to determine vitreous haze, sharp boundary edges can be ignored in order to avoid introducing artificially high spatial frequency content into the frequency domain of the images. Accordingly, a window function can be used to avoid the introduction of high spatial frequency content. In some embodiments, the window function selected for RQVHA may be a 2D Tukey window function.

FIG. 3C depicts the image of FIG. 3B with the contrast reversed and a window function applied to it. In this example embodiment, a 2D Tukey window function was applied with a parameter α=0.2. The transformation of FIG. 3C can be performed according to Equation (2) as reproduced below.

$${}^{C} I ( x , y ) = T ( x , y ) [ max \{ {}^{B} I ( x , y ) \} - {}^{B} I ( x , y ) ] \qquad ( 2 )$$

In Equation (2), ^{B}I(x, y) can represent the image after the green color channel is isolated and the resultant image is contrast limited adaptive histogram equalized and gamma corrected. T(x, y) represents the 2D Tukey window function. The resultant image after performing contrast reversal and applying a 2D Tukey window function is shown in FIG. 3C.

After the window function is applied, the RQVHA can include smoothing the image of FIG. 3C with a Gaussian smoothing function. Applying a Gaussian kernel can be advantageous for color fundus photographs that were captured in low light condition and therefore exhibit a low signal to noise ratio. Applying a Gaussian smoothing function is known to increase signal to noise ratio at the expense of lowering the resolution of the image. In addition, unlike the PQVHA methodology which involves setting a hard spatial frequency cutoff, applying a Gaussian kernel as a smoothing function can be considered as providing a Gaussian weighting to the spatial frequencies of the image. Accordingly, the RQVHA methodology does not require setting a hard spatial frequency cutoff.

FIG. 3D depicts the image of FIG. 3C after a Gaussian smoothing function has been applied to the image of FIG. 3C. The Gaussian smoothing function used in the RQVHA methodology can be given as Equation (3A) and/or Equation (3B) reproduced below.

$${}^{D} I ( x , y ) = \frac{1}{16 π ln 2 {F}_{1}^{2}} exp [ - \frac{{x}^{2} + {y}^{2}}{16 ln 1 {F}_{1}^{2}} ] * {}^{C} I ( x , y ) . \qquad ( 3 A )$$

$${}^{D} I ( x , y ) = \frac{ln 2}{4 π {F}_{1}^{2}} exp [ - ln 2 \frac{{x}^{2} + {y}^{2}}{4 {F}_{1}^{2}} ] * {}^{C} I ( x , y ) . \qquad ( 3 B )$$

In a preferred embodiment, in Equation (3A) and or Equation (3B), F₁=20 pixels to increase signal to noise ratio, although the value of F₁ can be varied according to some embodiments. For example, a value of F₁ can be selected between approximately 5 pixels and 100 pixels, depending on the level of artifacts present in the original color fundus photograph.

FIG. 3E shows the image of FIG. 3D after applying a Gaussian kernel high pass filter to the image of FIG. 3D. The Gaussian kernel high pass filter can be implemented according to Equation (4A) and/or Equation (4B), reproduced below.

$${}^{E} I ( x , y ) = {}^{D} I ( x , y ) - \frac{1}{16 π ln 2 {F}_{2}^{2}} exp [ - \frac{{x}^{2} + {y}^{2}}{16 ln 2 {F}_{2}^{2}} ] * {}^{D} I ( x , y ) . \qquad ( 4 A )$$

$${}^{E} I ( x , y ) = {}^{D} I ( x , y ) - \frac{ln 2}{4 π {F}_{2}^{2}} exp [ - ln 2 \frac{{x}^{2} + {y}^{2}}{4 {F}_{2}^{2}} ] * {}^{D} I ( x , y ) . \qquad ( 4 B )$$

In a preferred embodiment, a Gaussian kernel with Full Width Half Maximum (“FWHM”) of 50 pixels is used, although the FWHM can be varied from 20 to 200 in some embodiments, in order to remove slow-varying background signal. The use of a high-pass filter can eliminate low spatial frequencies corresponding to image artifacts, which would be less affected by blur, as well as background in the image, or residual differences due to varied lighting conditions across different color fundoscopic photographs. Previous methodologies, such as the PQVHA methodology employ a rectangle function which results in a sinc-weighting to spatial frequencies. Sinc-weighting has the unfavorable property of being non-monotonic, meaning certain higher spatial frequencies may be more suppressed than certain lower spatial frequencies, causing distortion of the image quality.

FIG. 3F depicts a magnitude spectrum as determined by calculating the absolute value of a transformation of the image of FIG. 3E from a spatial domain to a frequency domain, as can be achieved, for example, by performing a Fourier transformation of the image represented in FIG. 3E. By integrating over the magnitude spectrum, a clarify score L can be calculated. The haziness score H can be calculated according to Equation (5) below.

$$H = \frac{A}{L} + B , \qquad ( 5 )$$

In Equation (5), A and B are two constants that can be empirically determined to set the least hazy and most hazy color funduscopic photographs in each data set to 0 and 8, respectively, for comparison to the Miami scale for grading vitreous haze. This step could be modified to be compared to other scales, or arbitrarily transformed to match other distributions.

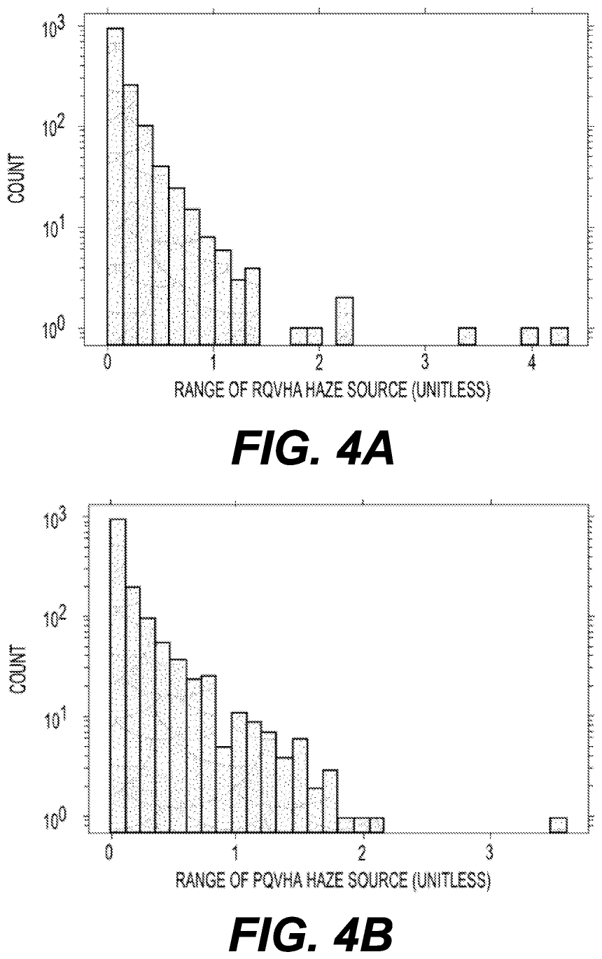

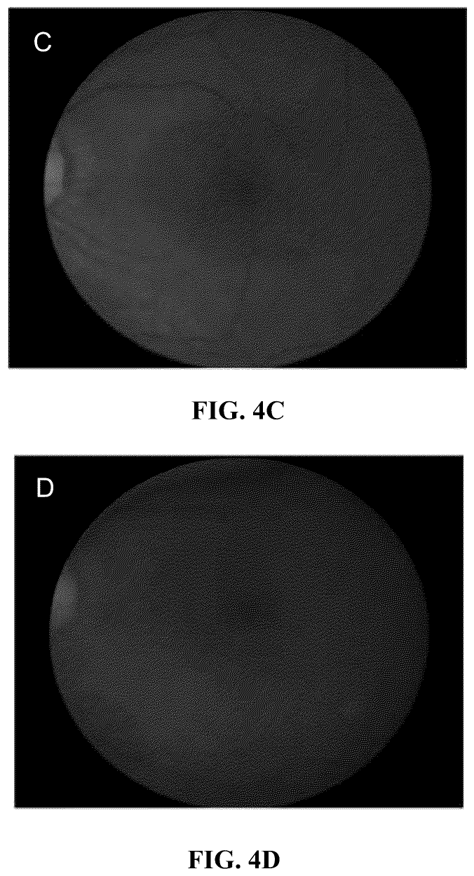

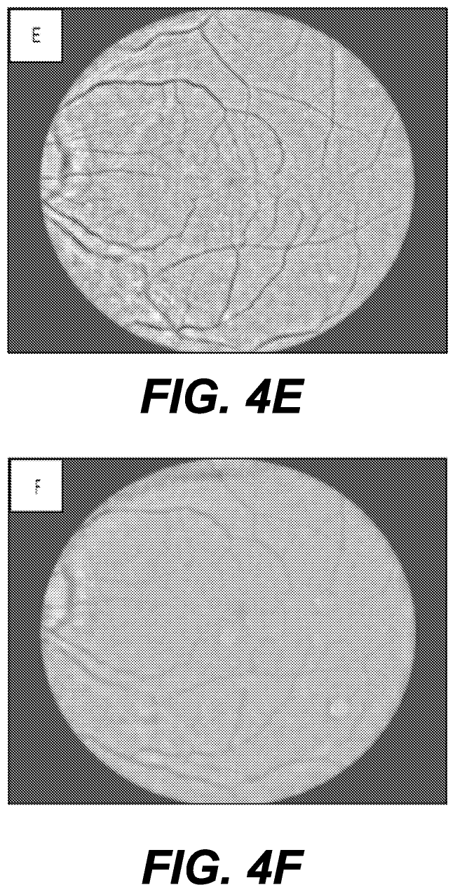

FIG. 4A and FIG. 4B shows a semi-log histogram of the coefficient of variation for RQHVA, and PQHVA, respectively, across color fundoscopic photographs corresponding to a respective eye of the same subject. More particularly, images from the same office visit of the same eye for a respective patient can be compared in order to minimized differences attributable to lighting conditions and differences attributable to camera settings that may be changed across different office visits. As can be seen when comparing the histogram of FIG. 4A with the histogram of FIG. 4B, the RQHVA methodology has substantially smaller number of cases in which the range of variation of calculated vitreous haze scores is large (i.e., above 1 point). The color fundoscopic photograph pairs (e.g., photographs from the same eye of a patient) that yielded the largest range of calculated vitreous haze are reproduced as FIGS. 4C and 4D. The RQHVA methodology calculated the vitreous haze of the color fundoscopic photograph in FIG. 4C as H=1.6, and the haze of the color fundoscopic photograph in FIG. 4D as H=5.9. Corresponding to the images of FIGS. 4C and 4D are the images of FIGS. 4E and 4F, respectively, except that FIGS. 4E and 4F have been processed according to Equation (1) for display purposes. As can be seen in FIG. 4F and corresponding FIG. 4D, the level of vitreous haze present in the color fundoscopic photograph is low, but the photograph appears hazy because the image is out of focus. Corresponding FIGS. 4E and 4C show images of the same eye and that there is very little haze present in the patient.

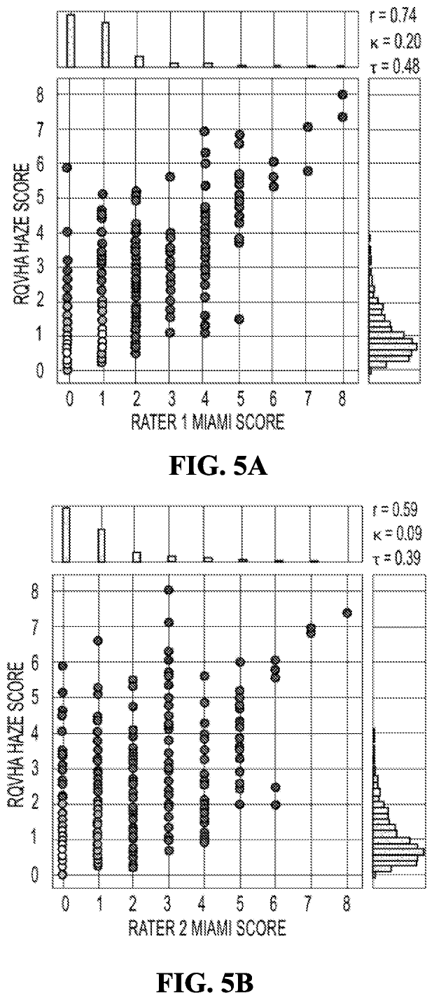

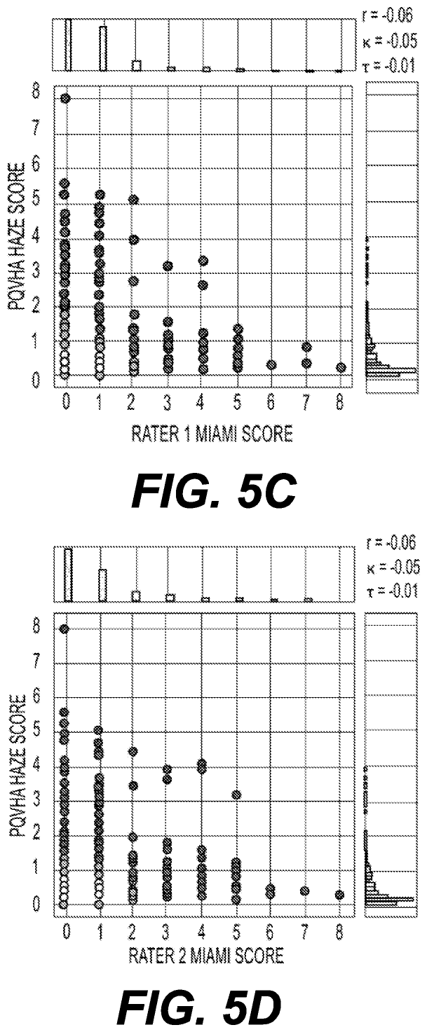

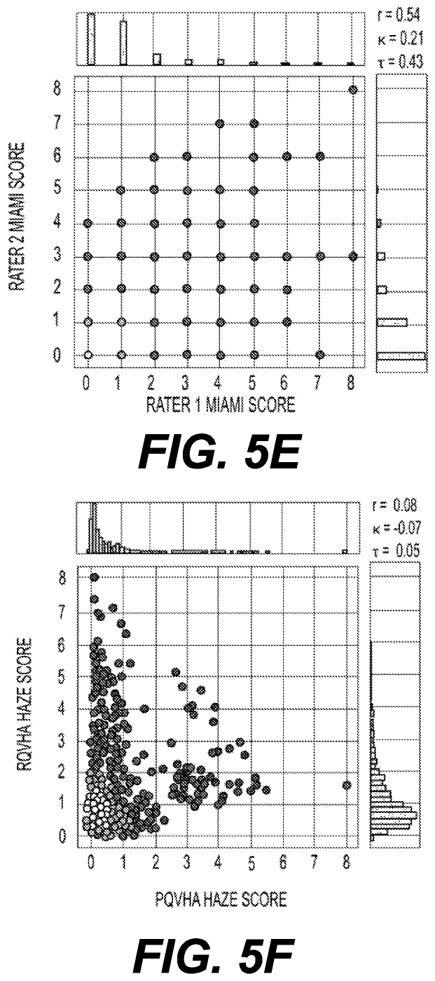

FIGS. 5A-5F depicts a histogram comparing the haze scores obtained by the RQVHA methodology and the PQVHA methodology to expert rater across all image sets. More specifically, 1377 image sets where two or more color funduscopic photographs were available for the same subject were used to determine how well the RQVHA methodology and the PQVHA methodology compared to expert raters of vitreous haze manually determining a haze score according to the Miami scale. For the comparison, a Pearson's correlation coefficient r, Kendall's coefficient τ, and Cohen's coefficient κ were calculated for each histogram. As can be seen by comparing FIGS. 5A and 5B to FIG. 5E, the RQVHA methodology produced similar agreement to first expert rater (FIG. 5A) and second expert rater (FIG. 5B) as did the first expert rater in comparison to the second expert rater (FIG. 5E). In contrast, the previously developed PQVHA methodology displayed no agreement with the first expert rater (FIG. 5C) and the second expert rater (FIG. 5D), with negative Pearson's coefficient, Kendall's coefficient, and Cohen's coefficient as shown in FIGS. 5C-5D. Similarly, there was no agreement between the RQVHA methodology and the PQVHA methodology, as shown in FIG. 5F.

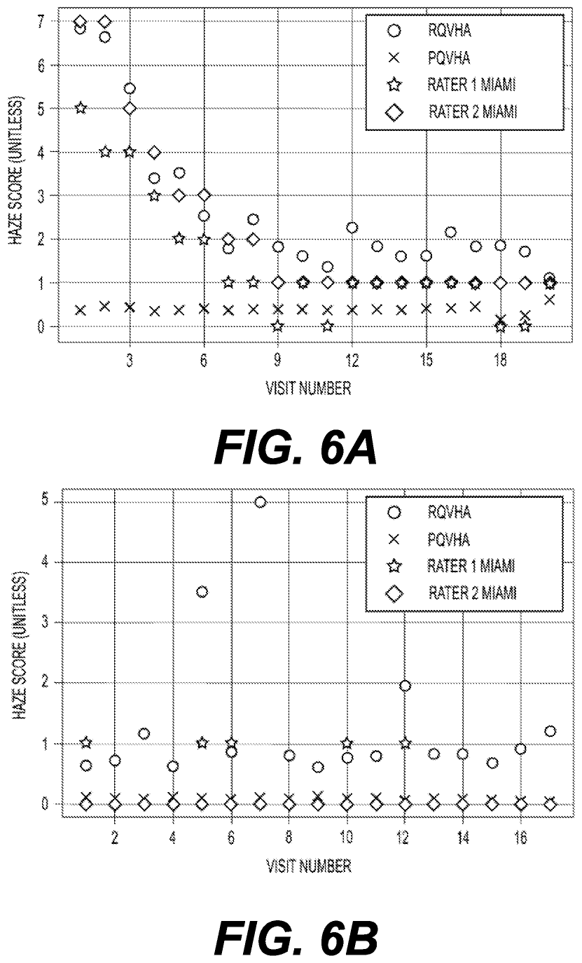

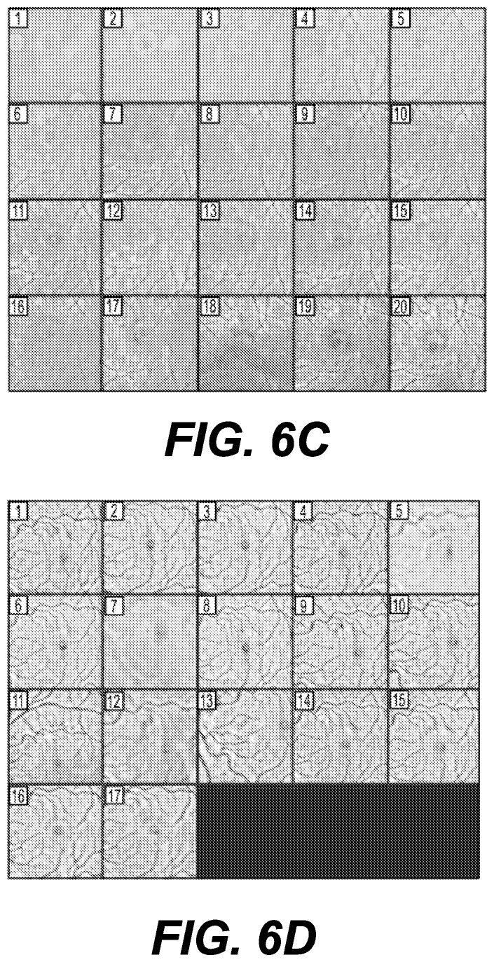

FIG. 6A depicts haze score variation for a patient that exhibited the largest decrease in haze over time for the RQVHA methodology, the PQVHA methodology, and as observed by both expert raters. FIG. 6C shows the sequence of color fundoscopic photographs correlating to FIG. 6A. As can be seen from the images of FIG. 6C, the large decrease in haze over time can largely be explained due to the blurriness of several of images in the time series.

FIG. 6B depicts haze score variation for a patient that exhibited the largest difference in haze over time for the RQVHA methodology, the PQVHA methodology, and as observed by both expert raters. FIG. 6D shows the sequence of color fundoscopic photographs correlating to FIG. 6B. Referring to the images of FIG. 6D, the largest disagreement between the RQVHA score can be attributed to image 7, in which both expert raters correctly rated the vitreous haze of the patient as 0, while the RQVHA methodology rated the vitreous haze as a 5 on the Miami scale. As can be seen, the image is very blurry, which may be attributed to either motion during image capture or the image being out of focus and is not attributable to vitreous haze in the patient's eye. A similar effect can be seen in images 5 and 12, and to a lesser degree image 3 of FIG. 6D, while other images (e.g., image 1, 9, 10, 13, etc.) that are not blurry or out of focus confirm that there is little to no vitreous haze present in the eye of this patient.

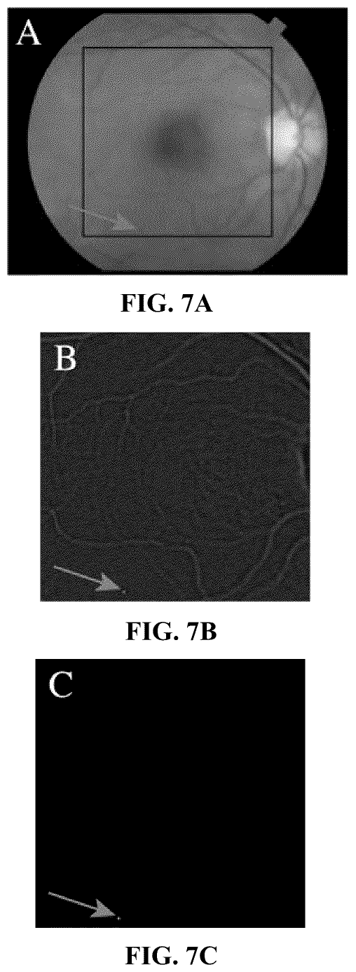

FIG. 7A depicts a raw color fundus photograph which includes an artifact corresponding to a dust particle on the lens of the camera, which lead to inaccurate vitreous haze grading by the PQVHA methodology as compared to the RQVHA methodology and the expert raters. FIG. 7B is the contrast-inverted and mean-filtered image corresponding to the raw color fundus photograph of FIG. 7A. FIG. 7C shows the local entropy of the image of FIG. 7B. As can be seen in FIG. 7C, very little detail is left in the image, and accordingly the PQVHA methodology inaccurately assigns a vitreous haze value of 8 to the image of FIG. 7A. This is because the PQVHA methodology uses local entropy to enhance fine spatial detail and reduce the effects of uneven lighting across the raw color fundus photograph. However, the drawback of using the local entropy is that applying a local entropy filter to images gives extremely high weight to small artifacts, such as dust particles as present in FIG. 7A. Accordingly, the presence of dust particles can wash out all detail present in the image. The RQVHA methodology does not rely on calculating local entropy to enhance spatial details and reduce the effects of uneven lighting. Instead, these issues can be addressed by isolating a color channel in a color fundoscopic photograph (e.g., the green color channel), and applying contrast limited histogram equalization and gamma correction. In contrast, both the expert raters one and two gave the image of FIG. 7A a vitreous haze score of 0 on the Miami scale, and the RQVHA methodology closely agreed with their assessments with a score of 1.6.

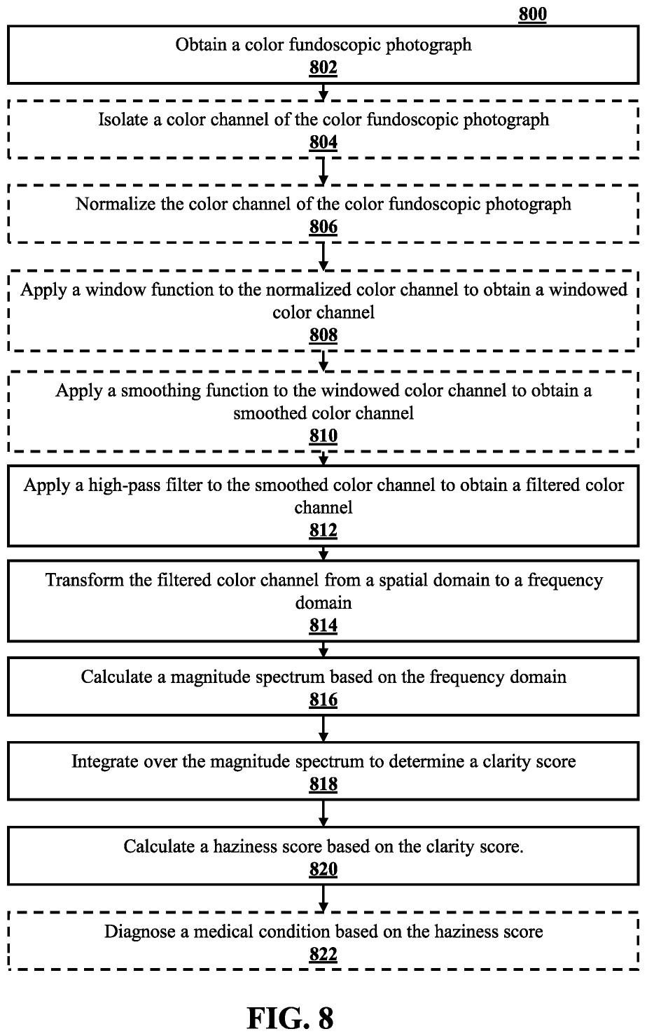

FIG. 8 is a flowchart of a method disclosed herein of calculating a haze score using the RQVHA methodology, in accordance with an example embodiment of the present disclosure. In step 802 of method 800, the method may include obtaining a color fundoscopic photograph. For example, vitreous haze grading platform 120 may receive a color fundoscopic photograph captured by fundus imaging device 130. In some embodiments, the color fundoscopic photograph may be stored on system 100, such as on database 118 and/or database 260 and may be transmitted to the vitreous haze grading platform 120 for analysis.

In optional step 804 of method 800, the method may include isolating a color channel of the color fundoscopic photograph. According to some embodiments, the green color channel of the color fundoscopic photograph may be isolated. The isolated green color channel may offer the benefit of providing higher contrast than traditional methods which involve transforming color fundus photographs into grayscale for analysis. According to some embodiments, the vitreous haze grading platform 120 may isolate the color channel from the color fundoscopic photograph.

In optional step 806, the method may include normalizing the color channel of the color fundoscopic photograph. In some embodiments, the normalization may include applying adaptive histogram equalization to the isolated color channel of the color fundoscopic image and applying a gamma correction function to the isolated color channel of the color fundoscopic image. Applying adaptive histogram equalization can further include applying contrast limited adaptive histogram equalization to the isolated color channel. According to some embodiments, the vitreous haze grading platform 120 may utilize processor(s) 210 to normalize the color channel of the color fundoscopic photograph.

In optional step 808, the method may include applying a window function to the normalized color channel to obtain a windowed color channel. For example, certain color fundoscopic images can include a macula that is on the edge of the image region, which can result in sharp edges at the boundary of the image. While previous methodologies, such as PQVHA attempt to overcome the issue of hard edges by cropping the image, the RQVHA methodology can include a windowing function to remove the sharp edges while retaining the entire image area of the color fundoscopic photograph. Various window functions can be used in the RQVHA methodology, including, for example, rectangular window functions, B-spline window functions, polynomial functions, sine window functions, cosine sum window functions, adjustable window functions, Hamming window functions, Hann window functions, Welch window functions, Planck-taper window functions, and hybrid window functions. In a preferred example, a 2D Tukey window function may be employed. In another preferred embodiment, a Planck-taper window function may be employed. According to some embodiments, the vitreous haze grading platform 120 may utilize processor(s) 210 to apply a window function to the normalized color channel.

In optional step 810, the method can include applying a smoothing function to the windowed color channel to obtain a smoothed color channel. When no window function is applied, optional step 810 can include applying a smoothing function to the color channel to obtain a smoothed color channel. For example, a gaussian function can be applied to the windowed color channel. The selected gaussian function can have a variable kernel size, for example, the kernel size of the gaussian smoothing function can be selected between 5 by 5 to 100 by 100 in some embodiments. In a preferred embodiment, the kernel size of the gaussian smoothing function can be 20 by 20 pixels. Other smoothing functions can be implemented in some embodiments. For example, simple average blurring, median filtering, or bilateral filtering can be implemented in lieu of gaussian blurring. According to some embodiments, the vitreous haze grading platform 120 may utilize processor(s) 210 to obtain the smoothed color channel. In some examples, the smoothing function can be, but is not limited to, a mean filter, a mean shift filter, a median filter, and/or a non-linear filter. Any smoothing function known in the art can be applied to the windowed color channel in optional step 810.

In step 812, the method can include applying a high-pass filter to the smoothed color channel to obtain a filtered color channel. When no smoothing function is applied, step 812 can include applying a high-pass filter to the normalized color channel to obtain a filtered color channel. According to some embodiments, the high-pass filter can be a gaussian filter. In a preferred embodiment, a full width half maximum parameter of 50 pixels can be used for the gaussian filter. According to some embodiments, the vitreous haze grading platform 120 may utilize processor(s) 210 to obtain the filtered color channel. In some examples, the smoothing function can be, but is not limited to, a Butterworth high-pass filter, and an ideal high pass filter, although any high-pass filter function known in the art can be applied to the smoothed color channel.

In step 814, the method can include transforming the filtered color channel from a spatial domain to a frequency domain. In a preferred embodiment, transforming the filtered color channel from a spatial domain to a frequency domain can include applying a Fourier transformation to the filtered color channel. According to some embodiments, the vitreous haze grading platform 120 may utilize processor(s) 210 to apply a Fourier transformation to the filtered color channel.

In step 816, the method can include calculating a magnitude spectrum to determine a clarity score. The magnitude spectrum can be calculated as an absolute value of the Fourier transformation of the filtered color channel. According to some embodiments, vitreous haze grading platform 120 can be configured to calculate the magnitude spectrum.

In step 818, the method can include integrating over the magnitude spectrum to determine a clarity score. For example, by integrating across all frequencies in a given magnitude spectrum, a clarity score can be determined. The clarity score can be inversely proportional to the haziness score, for example a haziness score between 0 and 8 as in the Miami scale for determining vitreous haze in fundoscopic photographs.

In step 820, the method can include calculating a haziness score based on the clarity score. The haziness score can be inversely proportional to the determined clarity score as given by Equation (5) discussed above. The constants A and B can be determined empirically for a given color fundoscopic photograph. The constants can be set such that the highest clarity photographs of a given set of fundoscopic photographs are scored 0 and the lowest clarity photographs of a given set of fundoscopic photographs are scored 8.

In optional step 822, the method can include diagnosing a medical condition based on the haziness score. The presence of vitreous haze can be indicative of one of variety of ocular disorders. For example, the diagnosed medical condition can include, but is not limited to the following conditions: uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, and/or reticulum cell sarcoma.

In some embodiments, the range of haze scores (i.e., maximum minus minimum) was calculated for both the PQVHA and RQVHA, for each of the 1377 image sets for which two or more images were available (86% of all image sets). Due to the large range for some image sets, which was likely the result of motion or unfocused images, the minimum haze score for each image set was used for subsequent analyses.

Due to the lack of ground truth, validation was investigated by comparison of the RQVHA values with the Miami scores from two expert raters across all 1639 image sets. For comparison, haze scores from the PQVHA were also compared to both raters. To quantify the comparison cosine similarity s (which is equivalent to Pearson's correlation coefficient discussed above except it does not have the unwanted property of being translation invariant), Kendall's τ, and Cohen's κ, after rounding to the nearest integer, were calculated across all images. It can be shown that, given the cosine similarity between two vectors, s_(a, b), the product of the cosine similarity between two other vectors is bounded by

$$\frac{1}{2} ( {s}_{a , b} - 1 ) \leq {s}_{b , c} {s}_{a , c} \leq \frac{1}{2} ( {s}_{a , b} + 1 ) . \qquad ( 6 )$$

This product metric is used assess the agreement of RQVHA and PQVHA with both raters simultaneously.

From a total 1377 image sets with 2 or more available images, the range of RQVHA was 0.14±0.28 (mean±standard deviation), with only 15 image sets having ≥1 point range. Similarly, the standard deviation was 0.10±0.19. These results indicate that a high degree of stability for the majority of image sets was achieved. For the PQVHA the range across all image sets was 0.06±0.16 and the standard deviation was 0.04±0.10, which is similarly stable although this is partially due to the tendency of the PQVHA to have very low haze values for our dataset.

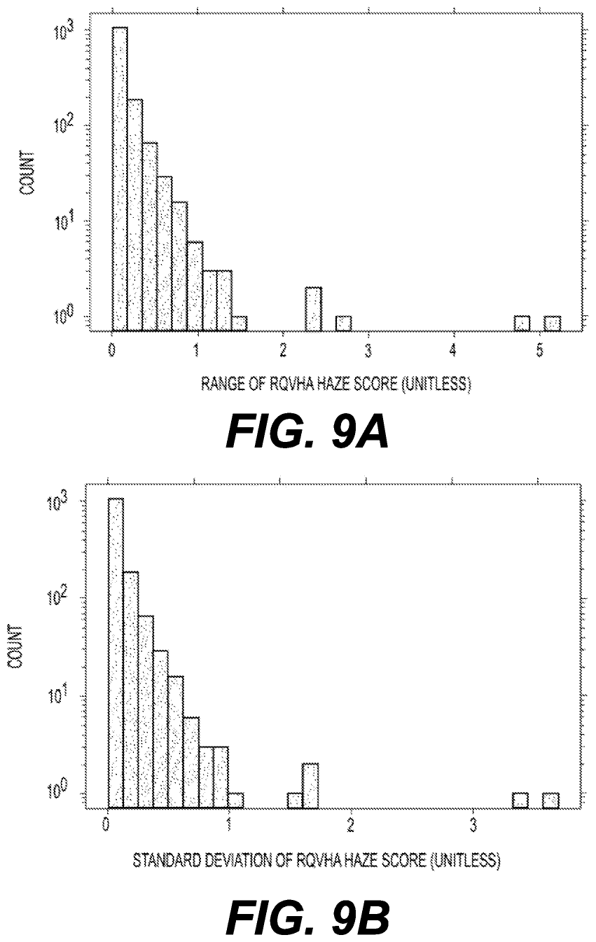

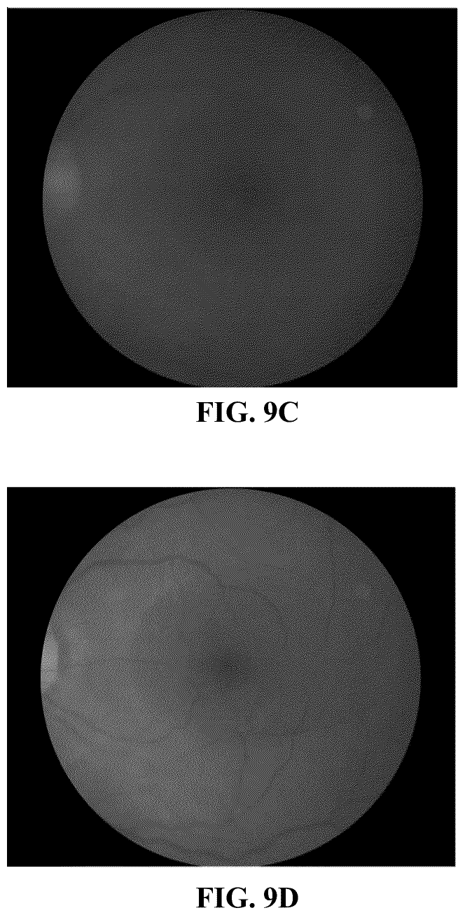

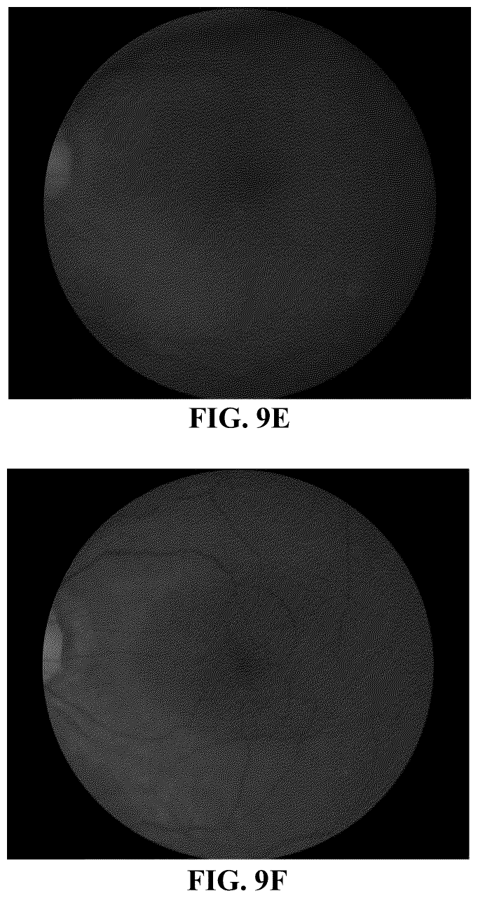

FIG. 9A shows a semi-log histogram of the range from the haze scores from RQHVA and FIG. 9B shows a semi-log histogram of the standard deviations from RQVHA within image sets. The largest range exhibited was 5.2 (max and min RQVHA scores of 7.9 and 2.7, respectively) as shown in FIG. 9A. The haze score of 7.9, however, was due to one image being out of focus, as can be seen in FIGS. 9C and 9D, indicating that this high variation is primarily due to the data quality, rather than a limitation of the RQVHA. The color fundoscopic photographs for the image sets (same eye, same session from a subject), which had the largest RQVHA range in FIGS. 9A and 9B are FIGS. 9C and 9D. The color fundoscopic photographs for the image sets which had the second largest RQVHA range in FIGS. 9A and 9B are FIGS. 9E and 9F. In particular, the RQVHA haze for FIGS. 9C and 9D are 7.9 and 2.7, respectively. The RQVHA haze for FIGS. 9E and 9F are 6.6. and 1.8, respectively. FIGS. 9C and 9E are out of focus and thus appear hazy, resulting in large haze scores, while successive images of FIGS. 9D and 9F are much sharper and thus have much lower haze scores. These results justify the use of using the minimum value from an image set and stress the importance of multiple images for reliable haze measurement.

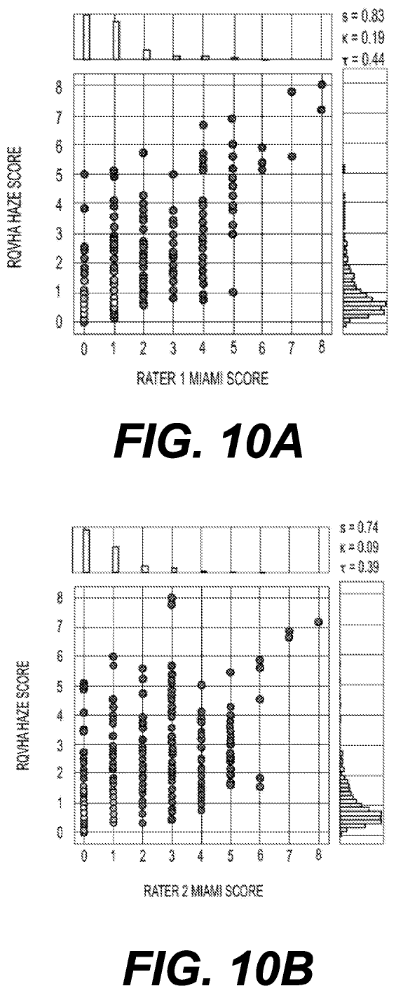

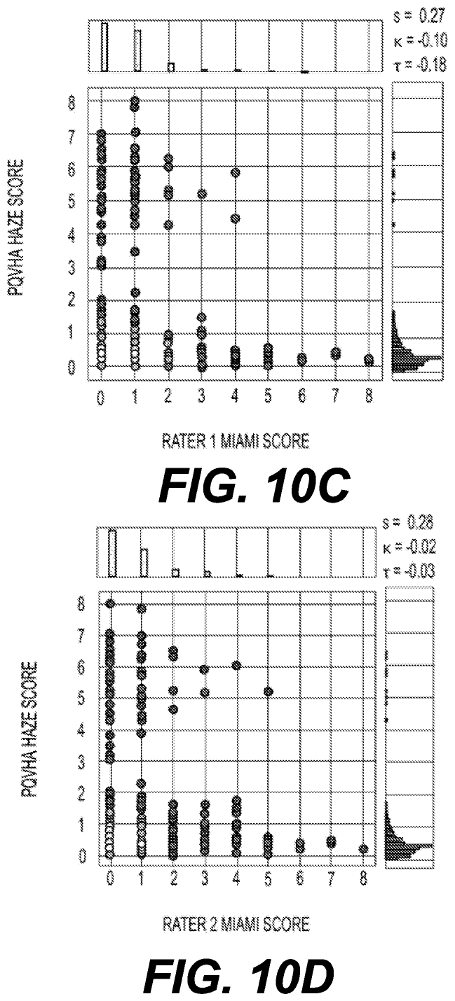

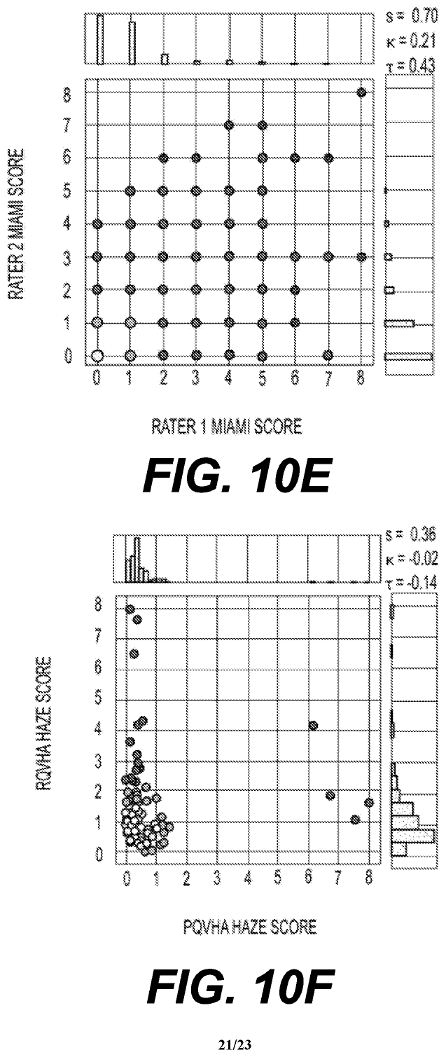

FIGS. 10A-10F shown histograms comparing the four different haze scores (PQVHA, RQVHA, Rater 1, and Rater 2) across all image sets using three comparison metrics (s, κ, and τ) are given in the top right of the corresponding histogram. Unlike FIGS. 5A-5C, FIGS. 10A-10C take into account cosine similarities. As shown, RQVHA has similar agreement with Rater 1 in (FIG. 10A) and Rater 2 (FIG. 10B) as they do to each other (FIG. 10E), while PQVHA has no agreement with either of the raters (FIG. 10C and FIG. 10D). Similarly, there is no agreement between PQVHA and RQVHA (FIG. 10F). RQVHA obtained similar agreement with Rater 1 (FIG. 10A) and Rater 2 (FIG. 10B) as they do to each other (FIG. 10E). Using Equation 6 and the cosine similarity between Rater 1 and Rater 2 (0.70) one can calculate the bound on the product of the cosine similarities of RQVHA with Rater 1 (s_(R,1)) and RQVHA with Rater 2 (s_(R,2)) to be −0.15≤s_(R,1) s_(R,2)≤0.85. Note that this is the same bound for PQVHA. The measured value for s_(R,1) s_(R,2) is 0.61. That s_(R,1) s_(R,2) is approaching the upper limit indicates good correspondence of RQVHA with both of the independent raters, with perfect correspondence being achieved, in this case 0.85, if the predicted haze score was equal to (Rater1+Rater2)/2. There was no agreement between PQVHA with either Rater 1 (FIG. 10C), Rater 2 (FIG. 10D) or with RQVHA (FIG. 10F). The product of the cosine similarities of the values from the Passaglia algorithm with the two raters, s_(P,1) s_(P,2) is 0.08. Since, s_(P,1) s_(P,2) is towards the lower limit, this indicates poor agreement with both of the raters. This is believed to be due to its unsuitability for the particular dataset at hand. It is important to mention that extreme vitreous haze is very rare, and even in our relatively large dataset there was only one image set which had a haze equal to 8 according to Rater 1 (FIG. 10C). There was a moderate correspondence with the two raters, obtaining cosine similarity values of 0.83 and 0.74 and Cohen's kappa values of 0.19 and 0.09. For comparison, the PQVHA obtained values of 0.27 and 0.28 for cosine similarity and −0.10 and −0.02 for Cohen's kappa.

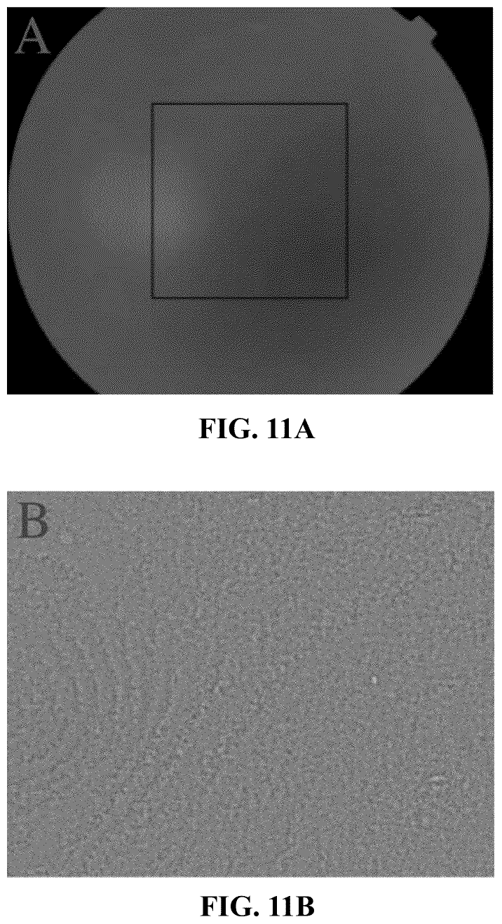

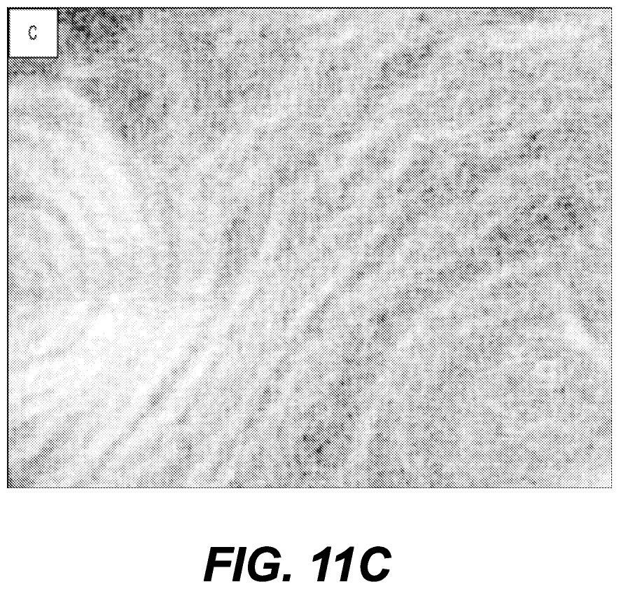

Unlike FIGS. 7A-7C, FIGS. 11A-11C 11A-11C do not include a dust particle. FIG. 11A is a raw fundus photograph of the subject which had the largest disagreement from RQVHA (8.0) and PQVHA (0.3). FIG. 11B shows a cropped grayscale, contrast-inverted and mean-filtered image corresponding to the black box in FIG. 11A. FIG. 11C shows the local entropy of FIG. 11B. As can be seen, the entropy filter in this case enhances non-physiological features, artificially reducing the haze. This image received an 8 from Rater 1 and a 3 from Rater 2. In some embodiments, local entropy, which was used by PQVHA, was not used because it was found to provide to enhance non-physiological features in the case of very hazy or low-illuminated images.

Examples of the present disclosure can be implemented according to at least the following clauses:

Clause 1: A method for grading vitreous haze, comprising: obtaining a color fundoscopic photograph; isolating a color channel of the color fundoscopic photograph; normalizing the color channel of the color fundoscopic photograph; applying a window function to the normalized color channel to obtain as windowed color channel; applying a smoothing function to the windowed color channel to obtain a smoothed color channel; applying a high-pass filter to the smoothed color channel to obtain a filtered color channel; transforming the filtered color channel from a spatial domain to a frequency domain; calculating a magnitude spectrum based on the frequency domain; integrating over the magnitude spectrum to determine a clarity score; and calculating a haziness score based on the clarity score.

Clause 2: The method of clause 1, wherein normalizing the color channel of the color fundoscopic photograph comprises: applying an adaptive histogram equalization to the color channel; and applying a gamma correction function to the color channel.

Clause 3: The method of clause 2, wherein the adaptive histogram equalization further comprises a contrast-limit adaptive histogram equalization.

Clause 4: The method of clause 1, wherein the window function comprises a Tukey window function.

Clause 5: The method of clause 1, wherein the smoothing function comprises a first gaussian filter.

Clause 6: The method of clause 1, wherein the high-pass filter comprises a second gaussian filter.

Clause 7: The method of clause 1, wherein transforming the filtered color channel from a spatial domain to a frequency domain comprises applying a Fourier transformation to the filtered color channel.

Clause 8: The method of clause 7, wherein calculating the magnitude spectrum comprises calculating an absolute value of the frequency domain.

Clause 9: The method of clause 1, wherein the color channel comprises a green channel.

Clause 10: The method of clause 1, further comprising diagnosing a medical condition based on the calculated haziness score.

Clause 11: The method of clause 10, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

Clause 12: A method for grading vitreous haze, comprising: obtaining a color fundoscopic photograph; isolating a color channel of the color fundoscopic photograph; normalizing the color channel of the color fundoscopic photograph; applying a high-pass filter to the smoothed color channel to obtain a filtered color channel; transforming the filtered color channel from a spatial domain to a frequency domain; calculating a magnitude spectrum based on the frequency domain; integrating over the magnitude spectrum to determine a clarity score; and calculating a haziness score based on the clarity score.

Clause 13: The method of clause 12, further comprising applying a window function to the normalized color channel.

Clause 14: The method of clause 12, further comprising applying a smoothing function to the normalized color channel to obtain a smoothed color channel.

Clause 15: The method of clause 12, wherein the color channel comprises a green channel.

Clause 16: The method of clause 12, wherein normalizing the color channel of the color fundoscopic photograph comprises: applying an adaptive histogram equalization to the color channel; and applying a gamma correction function to the color channel.

Clause 17: The method of clause 13, wherein the window function comprises a Tukey window function.

Clause 18: The method of clause 12, further comprising diagnosing a medical condition based on the calculated haziness score.

Clause 19: The method of clause 18, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

Clause 20: A system for grading vitreous haze, comprising: one or more processors; and a non-transitory memory storing instructions, wherein execution of the non-transitory memory storing instructions by the one or more processors cause the one or more processors to: obtain a color fundoscopic photograph; isolate a color channel of the color fundoscopic photograph; normalize the color channel of the color fundoscopic photograph; apply a window function to the normalized color channel to obtain a windowed color channel; apply a smoothing function to the windowed color channel to obtain a smoothed color channel; apply a high-pass filter to the smoothed color channel to obtain a filtered color channel; transform the filtered color channel from a spatial domain to a frequency domain; calculate a magnitude spectrum based on the frequency domain; integrate over the magnitude spectrum to determine a clarity score; and calculate a haziness score based on the clarity score.

Clause 21: The system of clause 20, wherein execution of the non-transitory memory storing instructions cause the one or more processors to: apply an adaptive histogram equalization to the color channel; and apply a gamma correction function to the color channel.

Clause 22: The system of clause 21, wherein the adaptive histogram equalization further comprises a contrast-limit adaptive histogram equalization.

Clause 23: The system of clause 20, wherein transforming the filtered color channel from a spatial domain to a frequency domain comprises applying a Fourier transformation to the filtered color channel.

Clause 24: The system of claim 20, wherein the color channel comprises a green channel.

Clause 25: The method of clause 20, further comprising diagnosing a medical condition based on the calculated haziness score.

Clause 26: The method of claim 25, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

Clause 27: A method for processing an image, comprising: obtaining a fundoscopic image; applying a window function to fundoscopic image to obtain a windowed fundoscopic image; applying a smoothing function to the windowed fundoscopic image to obtain a smoothed fundoscopic image; filtering the smoothed fundoscopic image with a high-pass filter to obtain a filtered fundoscopic image; transforming the filtered fundoscopic image from a spatial domain to a frequency domain; calculating a magnitude spectrum based on the frequency domain; and determining a haziness score of the fundoscopic image based on the calculated magnitude spectrum.

Clause 22: The method of clause 27, wherein the fundoscopic image further comprises a color fundoscopic image.

Clause 23: The method of clause 28, the method further comprising isolating a color channel of the color fundoscopic image.

Clause 24: The method of clause 29, the method further comprising normalizing the color channel of the color fundoscopic image.

Clause 25: The method of clause 30, wherein normalizing the color channel of the color fundoscopic photograph comprises: applying an adaptive histogram equalization to the color channel; and applying a gamma correction function to the color channel.

Clause 26: The method of clause 31, wherein the adaptive histogram equalization further comprises a contrast-limit adaptive histogram equalization.

Clause 27: The method of clause 27, wherein the window function comprises a Tukey window function.

Clause 28: The method of clause 27, wherein the smoothing function comprises a first gaussian filter.

Clause 29: The method of clause 27, wherein the high-pass filter comprises a second gaussian filter.

Clause 30: The method of clause 27, wherein transforming the filtered color channel from a spatial domain to a frequency domain comprises applying a Fourier transformation to the filtered color channel.

Clause 31: The method of clause 29, wherein the color channel comprises a green channel.

Clause 38: The method of clause 27, further comprising diagnosing a medical condition based on the determined haziness score.

Clause 39: The method of clause 38, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

The features and other aspects and principles of the disclosed embodiments may be implemented in various environments. Such environments and related applications may be specifically constructed for performing the various processes and operations of the disclosed embodiments or they may include a general-purpose computer or computing platform selectively activated or reconfigured by program code to provide the necessary functionality. Further, the processes disclosed herein may be implemented by a suitable combination of hardware, software, and/or firmware. For example, the disclosed embodiments may implement general purpose machines configured to execute software programs that perform processes consistent with the disclosed embodiments. Alternatively, the disclosed embodiments may implement a specialized apparatus or system configured to execute software programs that perform processes consistent with the disclosed embodiments. Furthermore, although some disclosed embodiments may be implemented by general purpose machines as computer processing instructions, all or a portion of the functionality of the disclosed embodiments may be implemented instead in dedicated electronics hardware.

The disclosed embodiments also relate to tangible and non-transitory computer readable media that include program instructions or program code that, when executed by one or more processors, perform one or more computer-implemented operations. The program instructions or program code may include specially designed and constructed instructions or code, and/or instructions and code well-known and available to those having ordinary skill in the computer software arts. For example, the disclosed embodiments may execute high level and/or low-level software instructions, such as machine code (e.g., such as that produced by a compiler) and/or high-level code that can be executed by a processor using an interpreter.

The technology disclosed herein typically involves a high-level design effort to construct a computational system that can appropriately process unpredictable data. Mathematical algorithms may be used as building blocks for a framework, however certain implementations of the system may autonomously learn their own operation parameters, achieving better results, higher accuracy, fewer errors, fewer crashes, and greater speed.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology described above with reference to user devices may include mobile computing devices. Those skilled in the art recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

In this description, numerous specific details have been set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features may alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to, and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for grading vitreous haze, comprising:
obtaining a color fundoscopic photograph;
isolating a color channel of the color fundoscopic photograph;
normalizing the color channel of the color fundoscopic photograph;
applying a window function to the normalized color channel to obtain a windowed color channel;
applying a smoothing function to the windowed color channel to obtain a smoothed color channel;
applying a high-pass filter to the smoothed color channel to obtain a filtered color channel;
transforming the filtered color channel from a spatial domain to a frequency domain;
calculating a magnitude spectrum based on the frequency domain;
integrating over the magnitude spectrum to determine a clarity score; and
calculating a haziness score based on the clarity score.

2. The method of claim 1, wherein normalizing the color channel of the color fundoscopic photograph comprises:
applying an adaptive histogram equalization to the color channel; and
applying a gamma correction function to the color channel.

3. The method of claim 2, wherein the adaptive histogram equalization further comprises a contrast-limit adaptive histogram equalization.

4. The method of claim 1, wherein the window function comprises a Tukey window function.

5. The method of claim 1, wherein the smoothing function comprises a first gaussian filter.

6. The method of claim 1, wherein the high-pass filter comprises a second gaussian filter.

7. The method of claim 1, wherein transforming the filtered color channel from a spatial domain to a frequency domain comprises applying a Fourier transformation to the filtered color channel.

8. The method of claim 7, wherein calculating the magnitude spectrum comprises calculating an absolute value of the frequency domain.

9. The method of claim 1, wherein the color channel comprises a green channel.

10. The method of claim 1, further comprising diagnosing a medical condition based on the calculated haziness score.

11. The method of claim 10, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

12. A method for grading vitreous haze, comprising:
obtaining a color fundoscopic photograph;
isolating a color channel of the color fundoscopic photograph;
normalizing the color channel of the color fundoscopic photograph;
applying a high-pass filter to the smoothed color channel to obtain a filtered color channel;
transforming the filtered color channel from a spatial domain to a frequency domain;
calculating a magnitude spectrum based on the frequency domain;
integrating over the magnitude spectrum to determine a clarity score; and
calculating a haziness score based on the clarity score.

13. The method of claim 12, further comprising applying a window function to the normalized color channel.

14. The method of claim 12, further comprising applying a smoothing function to the normalized color channel to obtain a smoothed color channel.

15. The method of claim 12, wherein the color channel comprises a green channel.

16. The method of claim 12, wherein normalizing the color channel of the color fundoscopic photograph comprises:
applying an adaptive histogram equalization to the color channel; and
applying a gamma correction function to the color channel.

17. The method of claim 13, wherein the window function comprises a Tukey window function.

18. The method of claim 12, further comprising diagnosing a medical condition based on the calculated haziness score.

19. The method of claim 18, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

20. A system for grading vitreous haze, comprising:
one or more processors; and
a non-transitory memory storing instructions, wherein execution of the non-transitory memory storing instructions by the one or more processors cause the one or more processors to:
obtain a color fundoscopic photograph;
isolate a color channel of the color fundoscopic photograph;
normalize the color channel of the color fundoscopic photograph;
apply a window function to the normalized color channel to obtain a windowed color channel;
apply a smoothing function to the windowed color channel to obtain a smoothed color channel;
apply a high-pass filter to the smoothed color channel to obtain a filtered color channel;
transform the filtered color channel from a spatial domain to a frequency domain;
calculate a magnitude spectrum based on the frequency domain;
integrate over the magnitude spectrum to determine a clarity score; and
calculate a haziness score based on the clarity score.

21. The system of claim 20, wherein execution of the non-transitory memory storing instructions cause the one or more processors to:
apply an adaptive histogram equalization to the color channel; and
apply a gamma correction function to the color channel.

22. The system of claim 21, wherein the adaptive histogram equalization further comprises a contrast-limit adaptive histogram equalization.

23. The system of claim 20, wherein transforming the filtered color channel from a spatial domain to a frequency domain comprises applying a Fourier transformation to the filtered color channel.

24. The system of claim 20, wherein the color channel comprises a green channel.

25. The system of claim 20, further comprising diagnosing a medical condition based on the calculated haziness score.

26. The system of claim 25, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

27. A method for processing an image, comprising:
obtaining a fundoscopic image;
applying a window function to fundoscopic image to obtain a windowed fundoscopic image;
applying a smoothing function to the windowed fundoscopic image to obtain a smoothed fundoscopic image;
filtering the smoothed fundoscopic image with a high-pass filter to obtain a filtered fundoscopic image;
transforming the filtered fundoscopic image from a spatial domain to a frequency domain;
calculating a magnitude spectrum based on the frequency domain; and
determining a haziness score of the fundoscopic image based on the calculated magnitude spectrum.

28. The method of claim 27, wherein the fundoscopic image further comprises a color fundoscopic image.

29. The method of claim 28, the method further comprising isolating a color channel of the color fundoscopic image.

30. The method of claim 29, the method further comprising normalizing the color channel of the color fundoscopic image.

31. The method of claim 30, wherein normalizing the color channel of the color fundoscopic photograph comprises:
applying an adaptive histogram equalization to the color channel; and
applying a gamma correction function to the color channel.

32. The method of claim 31, wherein the adaptive histogram equalization further comprises a contrast-limit adaptive histogram equalization.

33. The method of claim 27, wherein the window function comprises a Tukey window function.

34. The method of claim 27, wherein the smoothing function comprises a first gaussian filter.

35. The method of claim 27, wherein the high-pass filter comprises a second gaussian filter.

36. The method of claim 27, wherein transforming the filtered color channel from a spatial domain to a frequency domain comprises applying a Fourier transformation to the filtered color channel.

37. The method of claim 29, wherein the color channel comprises a green channel.

38. The method of claim 27, further comprising diagnosing a medical condition based on the determined haziness score.

39. The method of claim 38, wherein the medical condition is uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.

40. A method for diagnosing a medical condition of a patient eye, comprising:
obtaining a color fundoscopic photograph;
isolating a color channel of the color fundoscopic photograph;
normalizing the color channel of the color fundoscopic photograph;
applying a window function to the normalized color channel to obtain a windowed color channel;
applying a smoothing function to the windowed color channel to obtain a smoothed color channel;
applying a high-pass filter to the smoothed color channel to obtain a filtered color channel;
transforming the filtered color channel from a spatial domain to a frequency domain;
calculating a magnitude spectrum based on the frequency domain;
integrating over the magnitude spectrum to determine a clarity score;
calculating a haziness score based on the clarity score; and
diagnosing the medical condition based on the haziness score,
wherein the medical condition is one of uveitis, cataracts, macular degeneration, retinal vein occlusion, retinopathy, posterior vitreous detachment, retinal macroaneurysms, subarachnoid haemorrhages, ocular melanoma, retinis pigmentosa, retinal detachment, retinoblastoma neoplasia, leukemia, or reticulum cell sarcoma, and combinations thereof.
